(19) United States
(12) Patent Application Publication
(10) Pub. No.: US 2026/0021128 A1
(43) Pub. Date: Jan. 22, 2026

(54) COMPOSITION INCLUDING POLYENE ANTIFUNGAL DRUG AND USE THEREOF

(71) Applicant: JINAN UNIVERSITY, Guangzhou (CN)

(72) Inventors: Hong Zhang, Guangzhou (CN); Wencai Ye, Guangzhou (CN); Yishan Zhang, Guangzhou (CN); Minjing Cheng, Guangzhou (CN)

(21) Appl. No.: 19/344,467

(22) Filed: Sep. 29, 2025

Related U.S. Application Data
(63) Continuation-in-part of application No. PCT/CN2023/137318, filed on Dec. 8, 2023.

(30) Foreign Application Priority Data
Mar. 29, 2023 (CN) 202310317611.8

(51) Int. Cl. A61K 31/7048; A61K 31/592; A61K 31/593; A61P 31/10

(52) U.S. Cl. CPC A61K 31/7048; A61K 31/592; A61K 31/593; A61P 31/10

(57) ABSTRACT

A pharmaceutical composition for preventing and treating diseases caused by fungi is disclosed, which includes a polyene antifungal drug or a pharmaceutically acceptable salt thereof and vitamin D or an analogue thereof. The mass ratio of the polyene antifungal drug to vitamin D is 600:1 to 5:1, preferably 250:1 to 25:1. The pharmaceutical composition has excellent antifungal activity, and its active ingredients have good synergistic effects.

### CROSS-REFERENCE TO RELATED APPLICATIONS

The present application is a Continuation-in-part Application of PCT application No. PCT/CN2023/137318 filed on Dec. 8, 2023, which claims the benefit of Chinese Patent Application No. 202310317611.8 filed on Mar. 29, 2023. The contents of the above-identified applications are hereby incorporated by reference.

### TECHNICAL FIELD

The present application belongs to the field of medical technology, and specifically relates to a pharmaceutical composition for preventing and treating diseases caused by fungi. The pharmaceutical composition includes a polyene antifungal drug or a pharmaceutically acceptable salt thereof and vitamin D or an analogue thereof.

### BACKGROUND

Fungal infections can be divided into superficial fungal diseases and deep fungal diseases according to parts of a human body that are invaded. The main pathogens of superficial fungal infection are dermatophytes, Malassezia, and Candida, affecting more than 1 billion people each year. The main pathogens of invasive fungal infection are opportunistic fungi such as Candida, Aspergillus, Mucor, and Cryptococcus. Invasive fungal infections can affect more than 13 million people and kill 1.6 million people each year, exceeding the death toll from malaria, tuberculosis, and breast cancer. Even with standard antifungal treatment, immunosuppressed patients with deep fungal diseases face a mortality rate as high as 40-70%.

Fungal infections are mainly treated with drugs, but the available options are very limited. The available drugs are mainly azoles (fluconazole, voriconazole, etc.), echinocandins (caspofungin, micafungin, etc.), and polyenes (amphotericin B). Amphotericin B is an irreplaceable antifungal drug, characterized by its potent efficacy, broad spectrum of activity, and low resistance rates in the treatment of invasive fungal infections. Especially for those who are resistant to azoles and echinocandins, amphotericin B is often used as the last defense. However, the high incidence of dose-dependent nephrotoxicity and infusion-related adverse reactions limits the clinical application of amphotericin B. How to enhance the efficacy and reduce the toxicity of amphotericin B is one of the key tasks in the research and development of antifungal drugs.

At present, a variety of molecules that have been reported to enhance the efficacy of polyene amphotericin B and nystatin include plant-derived small molecule compounds such as polyphenol curcumin, berberine, allicin, acteoside, plumbagin, etc.; microbial extracts such as phialotide F, nectriatide, simpotentin; and marketed drugs such as rifapentine and fingolimod. However, most of these molecules only enhance the effect of amphotericin B in a narrow concentration range in vitro, and their safety and efficacy in vivo are unclear, so they cannot be used directly in clinical settings.

To reduce toxicity, new lipid formulations of amphotericin B have been developed, including liposomal amphotericin B, amphotericin B lipid complexes, and amphotericin B colloidal dispersion (ABCD). These formulations are less toxic but more expensive. This high cost renders these formulations particularly inaccessible for patients in developing countries, where more patients are infected with invasive fungal diseases and the mortality is higher. Furthermore, the reduced toxicity of liposomal amphotericin B is achieved by reducing its distribution in the kidneys. However, the kidney is often the organ with the highest fungal load in invasive fungal infections. Therefore, larger doses of liposomal amphotericin B are often required to achieve the same clearance of intrarenal pathogens.

It can be seen that existing antifungal drugs have problems such as large toxic side effects, limited clinical use, and lack of drug compositions. There is still a need to find safe and effective ways to increase the efficacy and reduce the toxicity of amphotericin B to expand its use and increase the survival rate of patients with deep fungal infections.

In view of this, the technical solution described in this application is provided.

### SUMMARY

An objective of this application is to provide an antifungal pharmaceutical composition that can effectively reduce a minimum inhibitory concentration of polyene antifungal drugs, increase its in vivo efficacy, and reduce its nephrotoxicity, thereby expanding applicable scope of polyene antifungal drugs and improving survival chances of patients with fungal infections.

Therefore, in a first aspect, this application provides an antifungal pharmaceutical composition comprising component A and component B, where component A is selected from a polyene antifungal drug or a pharmaceutically acceptable salt thereof; and component B is selected from vitamin D or an analogue thereof.

Preferably, the component A includes amphotericin B or nystatin, and the component B is selected from vitamin D₂, vitamin D₃, 25(OH)D, 1,25(OH)₂D, or eldecalcitol. The structural formulas of amphotericin B, nystatin, vitamin D₂, vitamin D₃, 25(OH)D, 1,25(OH)₂D, and eldecalcitol in this application are as follows:

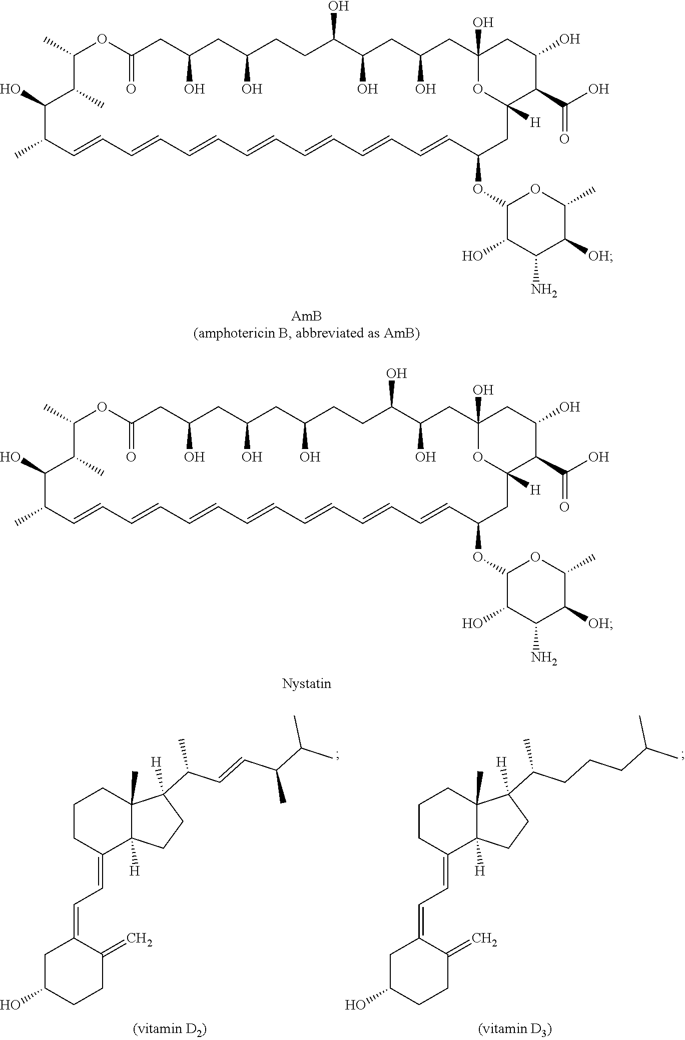

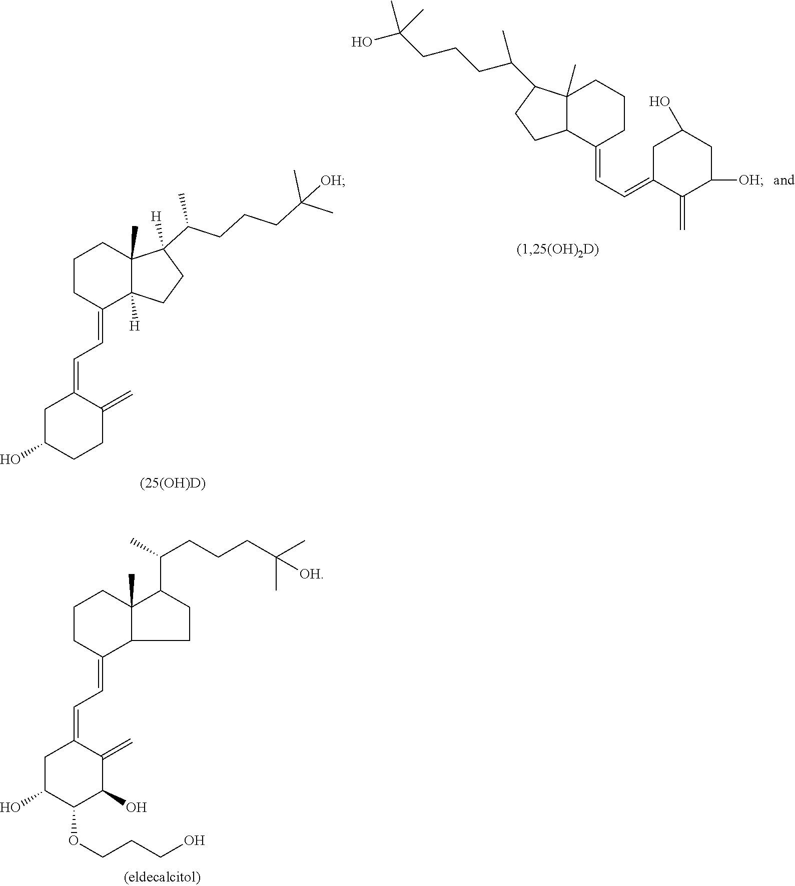 [SMILES: C=C1CC[C@H](O)C/C1=C/C=C1\CCC[C@]2(C)[C@@H]([C@H](C)CCCC(C)(C)O)CC[C@@H]12 ; C=C1/C(=C/C=C2\CCCC3(C)C2CCC3C(C)CCCC(C)(C)O)CC(O)CC1O ; C=C1/C(=C\C=C2/CCC[C@@]3(C)C2CCC3[C@H](C)CCCC(C)(C)O)C[C@@H](O)[C@@H](OCCCO)[C@@H]1O]

Preferably, the component A is amphotericin B, and the component B is vitamin D₃.

Preferably, a mass ratio of the polyene antifungal drug or a pharmaceutically acceptable salt thereof to vitamin D or an analogue thereof is 600:1 to 5:1, preferably 250:1 to 25:1.

The pharmaceutical composition in this application further includes one or more pharmaceutically acceptable carriers.

The one or more pharmaceutically acceptable carriers in this application refer(s) to one or more compatible solid or liquid fillers, diluents, or encapsulating substances suitable for administration to humans or other vertebrates; and the one or more pharmaceutically acceptable carriers also include(s) a natural or synthetic organic or inorganic component, which is combined with an active ingredient to facilitate application.

Specifically, the one or more pharmaceutically acceptable carriers in this application include(s) substances contained in a dosage form other than an active ingredient, including but not limited to liposomes, microcapsules, microspheres, micelles, nanoparticles, lipid complexes, colloidal dispersions, diluents, disintegrants, precipitation inhibitors, surfactants, flow aids, adhesives, lubricants, dispersants, suspending aids, isotonic agents, thickeners, emulsifiers, preservatives, stabilizers, hydrate agents, emulsification enhancers, buffering agents, absorbents, colorants, flavoring agents, sweeteners, corrigents, or antioxidants in the pharmaceutical field.

The dosage form can be an injection, a solution, a tablet, a capsule, a pastille, a pill, a troche, a gel, a syrup, a suspension, a tincture, a liniment, a spirit, a powder, an oil, a paste, an emplastrum, a film-forming agent, an aerosol, a lozenge, a patch, a nasal drop, an ear drop, a suppository, a lyophilized preparation of an active compound encapsulated in liposomes (within lipid layers or via liposomal encapsulation), or lipid complexes in an aqueous suspension.

This application provides a pharmaceutical composition including both a polyene antifungal drug or a pharmaceutically acceptable salt thereof and vitamin D or an analogue thereof in the form of a single pharmaceutical composition; or a combined form (e.g., a kit) including a separate formulation of a polyene antifungal drug or a pharmaceutically acceptable salt thereof and a separate formulation of vitamin D or an analogue thereof.

This application also provides a method for preparing the pharmaceutical composition. The method includes placing active ingredients of the pharmaceutical composition of this application in a pharmaceutically acceptable carrier in a certain proportion.

In another aspect, this application also provides the use of the above-mentioned pharmaceutical composition in the preparation of an antifungal drug.

Furthermore, the pharmaceutical composition is used for the prevention and/or treatment of a disease caused by fungi.

Preferably, the fungus is selected from Candida, Aspergillus, Histoplasma, Pneumocystis, Mucor, Cryptococcus, Cladophialophora, Trichophyton, or Microsporum.

Preferably, the fungus is selected from Candida albicans, Candida auris, Candida krusei, Aspergillus fumigatus, Histoplasma capsulatum, Pneumocystis carinii, Mucor, Cryptococcus neoformans, Cladophialophora carrionii, Trichophyton rubrum, or Microsporum canis.

Furthermore, the pharmaceutical composition in this application can be administered by injection, oral administration, or topical administration, depending on its dosage form.

The methods of injection administration include subcutaneous injection, intramuscular injection, intravenous injection, and intravenous drip.

The topical administration includes pulmonary inhalation administration, topical administration to a mucocutaneous surface, or topical administration to various tissue layers underlying the skin or mucosa.

In another aspect, the present application provides an anti-fungal method, including: administering an effective amount of component A and an effective amount of component B to a subject in need, the component A is selected from a polyene antifungal drug or a pharmaceutically acceptable salt thereof, the component B is selected from vitamin D or an analogue thereof; where the polyene antifungal drug is selected from amphotericin B or nystatin, and the vitamin D or an analogue thereof are selected from vitamin D₂, vitamin D₃, 25(OH)D, 1,25(OH)₂D, or eldecalcitol.

Compared with the shortcomings and deficiencies of the prior art, the present application has the following beneficial effects:
(1) In vitro, vitamin D or an analogue thereof can reduce the minimum inhibitory concentration of polyene antifungal drugs by 2-4 times.
(2) In Galleria mellonella larvae, the pharmaceutical composition can increase the survival rate of Galleria mellonella larvae infected with fungi by 15%-40% when compared with the use of polyene drugs alone.
(3) In the vulvovaginal region of mice infected with Candida, the pharmaceutical composition can reduce the vaginal fungal load of the mice to about 10² CFU/g when compared with the use of polyene drugs alone.
(4) In mice with systemic fungal infections, the pharmaceutical composition, when administered orally in combination with injection or by injection alone, can increase the survival rate of mice by 30%-40% and reduce the fungal load in the target organ (e.g., the kidney) to approximately 10² CFU/g when compared to the use of polyene drugs alone.
(5) In mice with systemic fungal infections, the pharmaceutical composition can reduce kidney damage in mice when compared with the use of polyene drugs alone, as shown by a decrease of about 60% in the creatinine and urea nitrogen levels.
(6) The mechanism by which the pharmaceutical composition increases the efficacy of polyene antifungal drugs is clear, and the mechanism is mainly to increase the content of ergosterol (i.e., the target of polyene antifungal drugs) in fungi, and enhance the binding ability of polyene antifungal drugs to ergosterol.

### BRIEF DESCRIPTION OF THE DRAWINGS

FIG. 1 shows survival rates of Candida albicans infected Galleria mellonella larvae treated with a pharmaceutical composition of vitamin D or an analogue thereof and polyenes.

FIG. 2 shows survival rates of Aspergillus fumigates infected Galleria mellonella larvae treated with a pharmaceutical composition of vitamin D or analogues thereof and polyenes.

FIG. 3 shows survival rates of Cryptococcus neoformans infected Galleria mellonella larvae treated with a pharmaceutical composition of vitamin D or an analogue thereof and polyenes.

FIG. 4 shows the efficacy of a pharmaceutical composition of vitamin D or an analogue thereof and amphotericin B in treating vaginal Candida infections.

FIG. 5 shows survival rates of systemic fungal infected mice treated with a combination of vitamin D or an analogue thereof and amphotericin B.

FIG. 6 shows the fungal load in the kidneys of systemic fungi infected mice treated with a combination of vitamin D or an analogue thereof and amphotericin B.

FIG. 7 shows plasma creatinine (CR) and blood urea nitrogen (BUN) levels of systemic fungi infected mice treated with a combination of vitamin D or an analogue thereof and amphotericin B.

FIG. 8 shows survival rates of systemic Candida albicans infected mice treated with a pharmaceutical composition of vitamin D and polyenes.

FIG. 9 shows survival rates of systemic Aspergillus fumigates infected mice treated with a pharmaceutical composition of vitamin D and polyenes.

FIG. 10 shows survival rates of systemic Cryptococcus neoformans infected mice treated with a pharmaceutical composition of vitamin D and polyenes.

FIG. 11 shows non-targeted metabolomics results of a pharmaceutical composition of vitamin D and polyenes for antifungal treatment.

FIG. 12 shows the expression of genes related to the ergosterol synthesis pathway in fungal cells when treated with a combination of vitamin D and polyenes, where “ns” represents no significance, “Acetyl-CoA” represents acetyl-coenzyme A, “Farnesyl-PP” represents farnesyl pyrophosphate, and “Rel. expression” represents relative expression.

FIG. 13 shows the ergosterol level in fungal cells when treated with a combination of vitamin D and polyenes, where “ns” represents no significance.

FIG. 14 shows the effect of vitamin D in combination with polyenes on the binding of polyenes to ergosterol.

### DETAILED DESCRIPTION

The present application is further described and explained in conjunction with examples below, but these examples are not intended to limit the scope of the present application.

Example 1 Preparation of Pharmaceutical Compositions of Polyene Antifungal Drugs and Vitamin D or Analogues thereof
(1) Preparation of storage solutions: Storage solutions (20 mg/ml) of vitamin D₂, vitamin D₃, 25(OH)D, 1,25(OH)₂D, and eldecalcitol were prepared using anhydrous ethanol, respectively. Storage solutions (20 mg/ml) of amphotericin B and nystatin were prepared using dimethyl sulfoxide (DMSO), respectively.
(2) Preparation method 1 of a composition: The storage solutions of vitamin D₂, vitamin D₃, 25(OH)D, 1,25(OH)₂D, eldecalcitol, amphotericin B, and nystatin were diluted 2-fold to a final concentration of 12.5 μg/ml to 1600 μg/ml. The diluted solutions were prepared into combination solutions according to a checkerboard method of drug combination. For instance, each concentration of vitamin D₃ was mixed with an equal volume of each concentration of amphotericin B to obtain a combination solution of vitamin D₃ (6.25 μg/ml)-amphotericin B (6.25 μg/ml), vitamin D₃ (6.25 μg/ml)-amphotericin B (12.5 μg/ml), vitamin D₃ (6.25 g/ml)-amphotericin B (25 μg/ml), vitamin D₃ (6.25 μg/ml)-amphotericin B (800 μg/ml), and so on until a combination solution of vitamin D₃ (800 μg/ml)-amphotericin B (800 μg/ml) was obtained.
(3) Preparation method 2 of a composition: Storage solutions of amphotericin B and nystatin were diluted with 0.5 wt % glucose solution to obtain dilute solutions A with a concentration of 0.1 mg/ml to 1.2 mg/ml. Storage solutions of vitamin D₂, vitamin D₃, 25(OH)D, 1,25(OH)₂D, and eldecalcitol were diluted to obtain dilute solutions B with a concentration of 2 g/ml or 20 μg/ml. Dilute solutions A and B were combined in a 1:1 volume ratio to obtain combined solutions. The final concentrations of the combined solutions were 0.05 mg/ml to 0.06 mg/ml of amphotericin B or nystatin, and 1 μg/ml and 10 μg/ml of vitamin D₂, vitamin D₃, 25(OH)D, 1,25(OH)₂D, or eldecalcitol. The final mass ratio of the polyene antifungal drug or a pharmaceutically acceptable salt thereof to vitamin D or an analogue thereof is 600:1 to 5:1. Further, the final mass ratio of the polyene antifungal drug or a pharmaceutically acceptable salt thereof to vitamin D or an analogue thereof is 250:1 to 25:1

### Example 2 Evaluation of the Antifungal Activity of Vitamin D or Analogues thereof in Combination with Amphotericin B in Vitro

### (1) Strains and Culture Media

Representative strains of Candida, Aspergillus, Histoplasma, Pneumocystis, Mucor, Cryptococcus, Cladophialophora, Trichophyton, or Microsporum were selected. Representative strains were numbered as follows: Candida albicans (SC5314, CA1-3), Candida auris (B11243, B11221), Candida krusei (CK1, CK2), Cryptococcus neoformans (ATCC 32609, CN2), Aspergillus fumigatus (ATCC 1022, AF17, AF23), Mucor (NRRL3631), Pneumocystis carinii (ATCC PRA-159), Histoplasma capsulatum (G217B), Cladophialophora carrionii (CBS114402), Trichophyton rubrum (ATCC4438), and Microsporum canis (ATCC36299). The strains were stored in yeast peptone dextrose (YPD)+glycerol media at −80° C., subcultured on YPD agar plates, and activated in YPD liquid media.

### (2) Experimental Method

The minimum inhibitory concentrations (MIC₅₀) of vitamin D₂, vitamin D₃, 25(OH)D, 1,25(OH)₂D, and eldecalcitol respectively in combination with amphotericin B against the above fungi were detected by the checkerboard method. The strains were cultured on yeast extract peptone dextrose (YEPD) agar media at 35° C. for 24 h. After the strains were activated twice, the activated strains were picked up with an inoculation loop. The selected strains were diluted with roswell park memorial institute (RPMI) 1640 culture medium and then counted using a cell counting plate. The concentrations of the fungal suspensions were adjusted to the required working concentration (2.0-2.5)×10³ CFU/ml. Before use, the fungal suspensions were prepared with a fresh culture medium. A drug storage solution was diluted to 2 times the working concentration with RPMI 1640 liquid medium. 100 μl of the diluted drug solution was taken, and after dilution in multiple proportions, the resulting solutions were added in one go to columns 2-11 of a 96-well plate. The first column of the 96-well plate was a growth control well (no drug was added, and 100 μl of RPMI 1640 liquid medium was used instead). The 12th column of the 96-well plate was a blank control well (only 200 μl of RPMI 1640 liquid medium was added). 100 μl of a fungal suspension was added to columns 1-11 of the 96-well plate to a working concentration. The plates with fungal solutions and drugs were cultured at 35° C. for 48 h, and the optical density (OD) values at a wavelength of 490 nm were measured using a microplate reader. The detection result of each well was subtracted by a background reading and compared with the result of a well containing a fungal suspension and without a drug. The minimum drug concentration that suppresses 50% of fungal growth was used as the final MIC₅₀ result. The fractional inhibitory concentration index (FICI) was calculated. FICI≤0.5 indicated synergism; FICI between 0.5 (exclusion of 0.5) and 4 indicated no interaction; and FICI>4 indicated antagonism. The above experiment was repeated 3 times.

### (3) Experimental Results

In this application, the antifungal activities of vitamin D₂, vitamin D₃, 25(OH)D, 1,25(OH)₂D, and eldecalcitol in combination with amphotericin B were determined by the methods described above, with the results shown in Tables 1-5. The synergism of the above-mentioned vitamin D and an analogue thereof on amphotericin B was commonly observed in Candida, Aspergillus, Histoplasma, Pneumocystis, Mucor, Cryptococcus, Cladophialophora, Trichophyton, and Microsporum. Among the above fungi, vitamin D and an analogue thereof can reduce the minimum inhibitory concentration of amphotericin B by 2-4 times.

TABLE 1
The in vitro antifungal MIC₅₀ (μg/ml) of the
combination of vitamin D₂ and amphotericin B (AmB)

| | | MIC₅₀ of use alone | | MIC₅₀ of combined use | | |
|---|---|---|---|---|---|---|
| | | (μg/ml) | | (μg/ml) | | |
| Strains | | Vitamin D₂ | AmB | Vitamin D₂ | AmB | FICI |
| Candida albicans | SC5314 | >64 | 1 | 1 | 0.5 | 0.5 |
| | CA1 | >64 | 1 | 1 | 0.5 | 0.5 |
| | CA2 | >64 | 1 | 1 | 0.5 | 0.5 |
| | CA3 | >64 | 0.5 | 2 | 0.125 | 0.25 |
| Candida auris | B11243 | >64 | 0.5 | 1 | 0.25 | 0.5 |
| | B11221 | >64 | 0.5 | 1 | 0.25 | 0.5 |
| Candida krusei | CK1 | >64 | 0.5 | 0.5 | 0.25 | 0.5 |
| | CK2 | >64 | 0.5 | 0.5 | 0.25 | 0.5 |
| Cryptococcus | ATCC 32609 | >64 | 1 | 1 | 0.25 | 0.25 |
| neoformans | CN2 | >64 | 1 | 1 | 0.25 | 0.25 |
| Aspergillus | ATCC 1022 | >64 | 1 | 2 | 0.5 | 0.5 |
| fumigatus | AF17 | >64 | 1 | 2 | 0.25 | 0.25 |
| | AF23 | >64 | 1 | 2 | 0.25 | 0.25 |
| Mucor | NRRL 3631 | >64 | 1 | 2 | 0.5 | 0.5 |
| Pneumocystis | ATCC | >64 | 1 | 1 | 0.5 | 0.5 |
| carinii | PRA-159 | | | | | |
| Histoplasma | G217B | >64 | 0.5 | 2 | 0.25 | 0.5 |
| capsulatum | | | | | | |
| Cladophialophora | CBS 114402 | >64 | 0.5 | 1 | 0.25 | 0.5 |
| carrionii | | | | | | |
| Trichophyton | ATCC4438 | >64 | 1 | 1 | 0.5 | 0.5 |
| rubrum | | | | | | |
| Microsporum canis | ATCC36299 | >64 | 1 | 1 | 0.5 | 0.5 |

TABLE 2
The in vitro antifungal MIC₅₀ (μg/ml) of the
combination of vitamin D₃ and amphotericin B (AmB)

| | | MIC₅₀ of use alone | | MIC₅₀ of combined use | | |
|---|---|---|---|---|---|---|
| | | (μg/ml) | | (μg/ml) | | |
| Strains | | Vitamin D₃ | AmB | Vitamin D₃ | AmB | FICI |
| Candida albicans | SC5314 | >64 | 1 | 0.5 | 0.5 | 0.5 |
| | CA1 | >64 | 1 | 1 | 0.5 | 0.5 |
| | CA2 | >64 | 1 | 1 | 0.5 | 0.5 |
| | CA3 | >64 | 0.5 | 2 | 0.125 | 0.25 |
| Candida auris | B11243 | >64 | 0.5 | 0.5 | 0.25 | 0.5 |
| | B11221 | >64 | 0.5 | 1 | 0.25 | 0.5 |
| Candida krusei | CK1 | >64 | 0.5 | 1 | 0.25 | 0.5 |
| | CK2 | >64 | 0.5 | 1 | 0.25 | 0.5 |
| Cryptococcus | ATCC 32609 | >64 | 1 | 1 | 0.25 | 0.25 |
| neoformans | CN2 | >64 | 1 | 1 | 0.25 | 0.25 |
| Aspergillus | ATCC 1022 | >64 | 1 | 1 | 0.5 | 0.5 |
| fumigatus | AF17 | >64 | 1 | 1 | 0.5 | 0.5 |
| | AF23 | >64 | 1 | 1 | 0.5 | 0.5 |
| Mucor | NRRL 3631 | >64 | 1 | 2 | 0.5 | 0.5 |
| Pneumocystis | ATCC | >64 | 1 | 2 | 0.5 | 0.5 |
| carinii | PRA-159 | | | | | |
| Histoplasma | G217B | >64 | 0.5 | 1 | 0.25 | 0.5 |
| capsulatum | | | | | | |
| Cladophialophora | CBS 114402 | >64 | 0.5 | 4 | 0.25 | 0.5 |
| carrionii | | | | | | |
| Trichophyton | ATCC4438 | >64 | 1 | 1 | 0.5 | 0.5 |
| rubrum | | | | | | |
| Microsporum canis | ATCC36299 | >64 | 1 | 1 | 0.5 | 0.5 |

TABLE 3
The in vitro antifungal MIC₅₀ (μg/ml) of the
combination of 25(OH)D and amphotericin B (AmB)

| | | MIC₅₀ of use alone | | MIC₅₀ of combined use | | |
|---|---|---|---|---|---|---|
| | | (μg/ml) | | (μg/ml) | | |
| Strains | | 25(OH)D | AmB | 25(OH)D | AmB | FICI |
| Candida albicans | SC5314 | >64 | 1 | 1 | 0.5 | 0.5 |
| | CA1 | >64 | 1 | 2 | 0.5 | 0.5 |
| | CA2 | >64 | 1 | 1 | 0.5 | 0.5 |
| | CA3 | >64 | 0.5 | 2 | 0.125 | 0.25 |
| Candida auris | B11243 | >64 | 0.5 | 0.5 | 0.25 | 0.5 |
| | B11221 | >64 | 0.5 | 0.5 | 0.25 | 0.5 |
| Candida krusei | CK1 | >64 | 0.5 | 0.5 | 0.25 | 0.5 |
| | CK2 | >64 | 0.5 | 0.5 | 0.25 | 0.5 |
| Cryptococcus | ATCC 32609 | >64 | 1 | 1 | 0.25 | 0.25 |
| neoformans | CN2 | >64 | 1 | 1 | 0.25 | 0.25 |
| Aspergillus | ATCC 1022 | >64 | 1 | 1 | 0.25 | 0.25 |
| fumigatus | AF17 | >64 | 1 | 1 | 0.25 | 0.25 |
| | AF23 | >64 | 1 | 1 | 0.25 | 0.25 |
| Mucor | NRRL 3631 | >64 | 1 | 1 | 0.5 | 0.5 |
| Pneumocystis carinii | ATCC | >64 | 1 | 1 | 0.5 | 0.5 |
| | PRA-159 | | | | | |
| Histoplasma | G217B | >64 | 0.5 | 1 | 0.25 | 0.5 |
| capsulatum | | | | | | |
| Cladophialophora | CBS 114402 | >64 | 0.5 | 2 | 0.25 | 0.5 |
| carrionii | | | | | | |
| Trichophyton rubrum | ATCC4438 | >64 | 1 | 1 | 0.5 | 0.5 |
| Microsporum canis | ATCC36299 | >64 | 1 | 1 | 0.5 | 0.5 |

TABLE 4
The in vitro antifungal MIC₅₀ (μg/ml) of the
combination of 1.25(OH)₂D and amphotericin B (AmB)

| | | MIC₅₀ of use alone | | MIC₅₀ of combined use | | |
|---|---|---|---|---|---|---|
| | | (μg/ml) | | (μg/ml) | | |
| Strains | | 1.25(OH)₂D | AmB | 1.25(OH)₂D | AmB | FICI |
| Candida albicans | SC5314 | >64 | 1 | 1 | 0.25 | 0.25 |
| | CA1 | >64 | 1 | 1 | 0.25 | 0.25 |
| | CA2 | >64 | 1 | 2 | 0.5 | 0.5 |
| | CA3 | >64 | 0.5 | 4 | 0.125 | 0.25 |
| Candida auris | B11243 | >64 | 0.5 | 0.5 | 0.25 | 0.5 |
| | B11221 | >64 | 0.5 | 0.5 | 0.25 | 0.5 |
| Candida krusei | CK1 | >64 | 0.5 | 0.5 | 0.25 | 0.5 |
| | CK2 | >64 | 0.5 | 0.5 | 0.25 | 0.5 |
| Cryptococcus | ATCC 32609 | >64 | 1 | 0.5 | 0.125 | 0.125 |
| neoformans | CN2 | >64 | 1 | 1 | 0.125 | 0.125 |
| Aspergillus | ATCC 1022 | >64 | 1 | 1 | 0.5 | 0.5 |
| fumigatus | AF17 | >64 | 1 | 1 | 0.5 | 0.5 |
| | AF23 | >64 | 1 | 1 | 0.5 | 0.5 |
| Mucor | NRRL 3631 | >64 | 1 | 1 | 0.25 | 0.25 |
| Pneumocystis | ATCC | >64 | 1 | 1 | 0.25 | 0.25 |
| carinii | PRA-159 | | | | | |
| Histoplasma | G217B | >64 | 0.5 | 1 | 0.25 | 0.5 |
| capsulatum | | | | | | |
| Cladophialophora | CBS 114402 | >64 | 0.5 | 2 | 0.25 | 0.5 |
| carrionii | | | | | | |
| Trichophyton | ATCC4438 | >64 | 1 | 1 | 0.5 | 0.5 |
| rubrum | | | | | | |
| Microsporum canis | ATCC36299 | >64 | 1 | 1 | 0.5 | 0.5 |

TABLE 5
The in vitro antifungal MIC₅₀ (μg/ml) of the
combination of eldecalcitol and amphotericin B (AmB)

| | | MIC₅₀ of use alone | | MIC₅₀ of combined use | | |
|---|---|---|---|---|---|---|
| | | (μg/ml) | | (μg/ml) | | |
| Strains | | Eldecalcitol | AmB | Eldecalcitol | AmB | FICI |
| Candida albicans | SC5314 | >64 | 1 | 1 | 0.5 | 0.5 |
| | CA1 | >64 | 1 | 1 | 0.5 | 0.5 |
| | CA2 | >64 | 1 | 1 | 0.5 | 0.5 |
| | CA3 | >64 | 0.5 | 4 | 0.125 | 0.25 |
| Candida auris | B11243 | >64 | 0.5 | 1 | 0.25 | 0.5 |
| | B11221 | >64 | 0.5 | 1 | 0.25 | 0.5 |
| Candida krusei | CK1 | >64 | 0.5 | 1 | 0.25 | 0.5 |
| | CK2 | >64 | 0.5 | 1 | 0.25 | 0.5 |
| Cryptococcus | ATCC 32609 | >64 | 1 | 1 | 0.25 | 0.25 |
| neoformans | CN2 | >64 | 1 | 1 | 0.25 | 0.25 |
| Aspergillus | ATCC 1022 | >64 | 1 | 1 | 0.5 | 0.5 |
| fumigatus | AF17 | >64 | 1 | 2 | 0.5 | 0.5 |
| | AF23 | >64 | 1 | 2 | 0.5 | 0.5 |
| Mucor | NRRL 3631 | >64 | 1 | 4 | 0.5 | 0.5 |
| Pneumocystis | ATCC | >64 | 1 | 1 | 0.5 | 0.5 |
| carinii | PRA-159 | | | | | |
| Histoplasma | G217B | >64 | 0.5 | 2 | 0.25 | 0.5 |
| capsulatum | | | | | | |
| Cladophialophora | CBS 114402 | >64 | 0.5 | 1 | 0.25 | 0.5 |
| carrionii | | | | | | |
| Trichophyton | ATCC4438 | >64 | 1 | 1 | 0.5 | 0.5 |
| rubrum | | | | | | |
| Microsporum canis | ATCC36299 | >64 | 1 | 1 | 0.5 | 0.5 |

### Example 3 Evaluation of the In Vitro Antifungal Activity of Vitamin D or Analogues Thereof in Combination with Nystatin

The in vitro antifungal activity of the combination of vitamin D and nystatin was evaluated using the interaction between vitamin D₃ and nystatin as an example.

### (1) Experimental Method

The experimental method was the same as the experimental method described in Example 2.

### (2) Experimental Results

The antifungal activity of vitamin D₃ and nystatin in this application is shown in Table 6. The synergism of vitamin D on nystatin is common in Candida, Aspergillus, Histoplasma, Pneumocystis, Mucor, Cryptococcus, Trichophyton, Microsporum, and Cladophialophora. In the above-mentioned fungi, vitamin D and analogues thereof can reduce the minimum inhibitory concentration of nystatin by 2-4 times.

TABLE 6
In vitro antifungal MIC₅₀ (μg/ml) of the combination of vitamin D₃ and nystatin

| | | MIC₅₀ of use alone | | MIC₅₀ of combined use | | |
|---|---|---|---|---|---|---|
| | | (μg/ml) | | (μg/ml) | | |
| Strains | | Vitamin D₃ | Nystatin | Vitamin D₃ | Nystatin | FICI |
| Candida albicans | SC5314 | >64 | 2 | 4 | 0.5 | 0.25 |
| | CA1 | >64 | 4 | 4 | 1 | 0.25 |
| | CA2 | >64 | 4 | 4 | 2 | 0.5 |
| | CA3 | >64 | 4 | 8 | 1 | 0.25 |
| Candida auris | B11243 | >64 | 8 | 0.5 | 4 | 0.5 |
| | B11221 | >64 | 8 | 0.5 | 2 | 0.25 |
| Candida krusei | CK1 | >64 | 4 | 0.5 | 2 | 0.5 |
| | CK2 | >64 | 4 | 0.5 | 2 | 0.5 |
| Cryptococcus | ATCC 32609 | >64 | 4 | 0.5 | 1 | 0.25 |
| neoformans | CN2 | >64 | 4 | 1 | 1 | 0.25 |
| Aspergillus fumigatus | ATCC 1022 | >64 | 4 | 1 | 1 | 0.25 |
| | AF17 | >64 | 4 | 2 | 2 | 0.5 |
| | AF23 | >64 | 4 | 1 | 2 | 0.5 |
| Mucor | NRRL 3631 | >64 | 4 | 1 | 2 | 0.5 |
| Pneumocystis carinii | ATCC | >64 | 4 | 1 | 2 | 0.5 |
| | PRA-159 | | | | | |
| Histoplasma | G217B | >64 | 4 | 1 | 2 | 0.5 |
| capsulatum | | | | | | |
| Cladophialophora | CBS 114402 | >64 | 4 | 2 | 2 | 0.5 |
| carrionii | | | | | | |
| Trichophyton rubrum | ATCC4438 | >64 | 1 | 1 | 0.5 | 0.5 |
| Microsporum canis | ATCC36299 | >64 | 1 | 1 | 0.5 | 0.5 |

### Example 4 Efficacy of a Pharmaceutical Composition of Vitamin D or Analogues Thereof and Polyenes in Treating Galleria mellonella Infected with Fungi

Taking common clinical fungi Candida albicans, Aspergillus fumigatus, and Cryptococcus neoformans as examples, the synergistic activity of vitamin D or analogues thereof on polyene antifungal drugs was evaluated in Galleria mellonella larvae.

### (1) Experimental Method

First, the last instar larvae of Galleria mellonella larvae (about 3 cm in length and 0.5 g in weight) were selected. Larvae with healthy bodies and without a black spot were selected as much as possible. The selected larvae were randomly divided into groups, with 20 larvae in each group, and each group of larvae were placed in a dish with a diameter of 10 cm. Candida albicans SC5314, Aspergillus fumigatus AF17, and Cryptococcus neoformans ATCC 32609 were activated in yeast peptone dextrose (YPD) liquid medium until the end of the logarithmic growth phase. The activated fungal cells were collected and then washed twice with phosphate buffered saline (PBS). A fungal solution concentration was adjusted to 1×10⁸ colony-forming unit (CFU)/ml with normal saline. The Galleria mellonella larvae were selected and fixed with the abdomens facing upwards. The injection sites of Galleria mellonella larvae were disinfected with alcohol. A larvae body was held by hand, and a Hamilton microinjection needle was used to gently pierce the second leg at the lower right of the worm body. 10 μl of fungal solution was injected from the needle tip to the middle of the second leg. After the model was successfully established, the larvae body was placed on a clean paper towel and allowed to stand for 1 min. 10 μl of a drug was injected into the second lower left leg of the larvae body. The injected drug was vitamin D₂, vitamin D₃, 25(OH)D, 1,25(OH)₂D, eldecalcitol, amphotericin B, nystatin, or a mixture of the above vitamin D or an analogue thereof with amphotericin B or nystatin. The concentrations of vitamin D₂ and vitamin D₃ were 1 μg/kg, respectively. The concentrations of 25(OH)D, 1,25(OH)₂D, and eldecalcitol were 0.5 μg/kg, respectively. The concentration of amphotericin B was 0.5 mg/kg. The concentration of nystatin was 1 mg/kg. The concentrations of these drugs when used alone were consistent with the concentrations of these drugs in the compositions. The infection control group was injected with the same volume of normal saline. The Galleria mellonella larvae were observed regularly every day and dead larvae bodies were removed. The survival time of Galleria mellonella larvae in each group was recorded, and the survival rates of Galleria mellonella larvae were calculated after 3 days.

### (2) Experimental Results

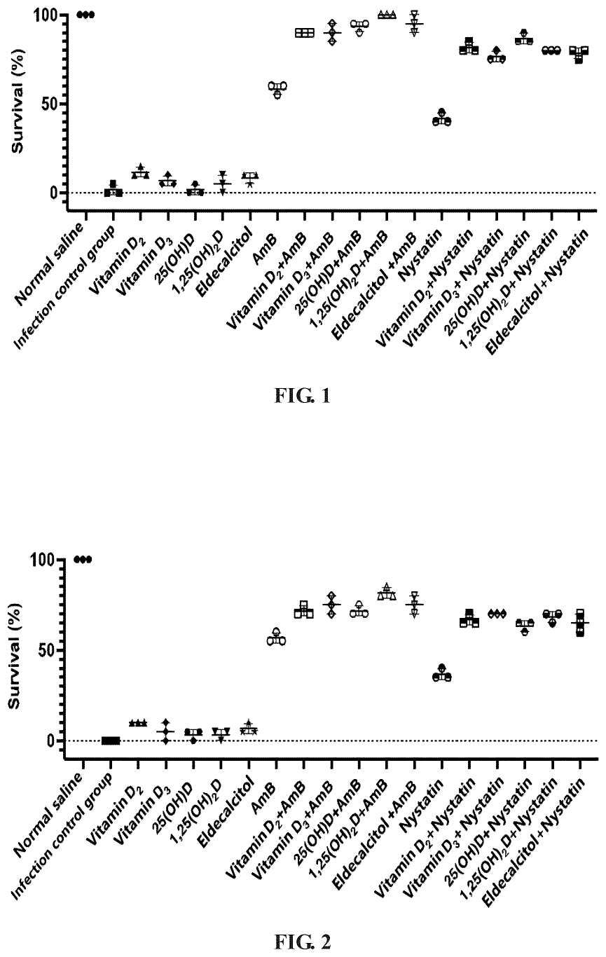

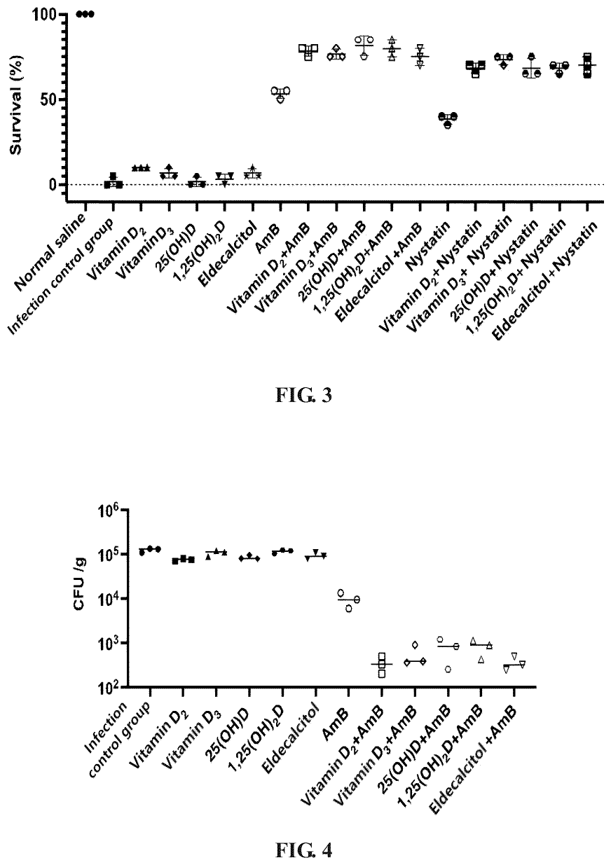

The experimental results are shown in FIGS. 1-3. All Galleria mellonella larvae in the control group (WT, i.e., wild-type (WT) infected Galleria mellonella larvae) died within fourth day after being infected with Candida albicans, Aspergillus fumigatus, or Cryptococcus neoformans. Single injection of vitamin D₂, vitamin D₃, 25(OH)D, 1,25(OH)₂D, and eldecalcitol has no significant effect on the survival rates of Galleria mellonella larvae. The survival rate of Galleria mellonella larvae is increased by 50%-70% through single injection of amphotericin B. The combination of vitamin D₂, vitamin D₃, 25(OH)D, 1,25(OH)₂D, or eldecalcitol, with amphotericin B or nystatin can increase the survival rates of Galleria mellonella larvae to 100% (P<0.05).

### Example 5 Efficacy of a Pharmaceutical Composition of Vitamin D or Analogues Thereof and Amphotericin B in the Treatment of Vaginal Candida Infections

### (1) Experimental Method

Three days before infection, 18-20 g female BALB/c mice were intraperitoneally injected with 100 μl of estrogen (0.5 mg β-estradiol in 100 μl of sesame oil) and 100 μl of cyclophosphamide (100 mg/kg). The injection was repeated once every three days after the initial injection. On the day of infection, Candida albicans SC5314 activated overnight in liquid YPD was collected. The collected Candida albicans SC5314 was washed twice with PBS and the concentration of SC5314 solution was adjusted to 1×10⁷ CFU/ml. Mice were anesthetized with chloral hydrate. 10 μl of a fungal solution was injected into the vaginal cavity of each mouse, and mouse's hips were raised and left to stand for half an hour. On the second day after infection, the corresponding groups (3 mice in each group) were administered vitamin D₂, vitamin D₃, 25(OH)D, 1,25(OH)₂D, eldecalcitol, amphotericin B, or a pharmaceutical composition of the above vitamin D or analogues thereof respectively and amphotericin B (prepared according to the composition preparation method 2 of Example 1). The concentrations of vitamin D₂, vitamin D₃, 25(OH)D, 1,25 (OH)₂D, and eldecalcitol were all 0.5 mg/ml. The concentration of amphotericin B was 0.5 mg/kg. The concentrations of these drugs when used alone were consistent with the concentrations of these drugs in the compositions. The administration of these drugs and compositions was conducted for 3 consecutive days. After administration, the mouse's hips were kept elevated and allowed to stand for half an hour. On the fourth day, the vaginas of the mice were irrigated with PBS, and a resulting eluate was diluted and spread on a YPD plate for culture for 48 h, after which the fungi on the plate were counted.

### (2) Experimental Results

As shown in FIG. 4, the use of vitamin D₂, vitamin D₃, 25(OH)D, 1,25(OH)₂D, and eldecalcitol alone has no significant effect on the vaginal fungal load in mice. Compared with the use of amphotericin B alone, the pharmaceutical composition reduces the vaginal fungal load in mice to approximately 10² CFU/g (P<0.05).

### Example 6 Efficacy and Toxicity of Vitamin D or Analogues Thereof in Combination with Polyene Drugs in the Treatment of Mice with Systemic Fungal Infections

### (1) Experimental Method

Candida albicans SC5314 was activated in YPD liquid medium until the end of logarithmic growth phase. The fungal cells were collected and then washed twice with PBS. The concentration of the fungal solution was adjusted to 2×10⁶ CFU/ml with normal saline. Female BALB/c mice weighing 18-22 g were randomly divided into groups with 10 mice in each group. Mice were grouped into NS (injected with normal saline), WT (injected with SC5314), WT+vitamin D₂, WT+vitamin D₃, WT+25(OH)D, WT+1,25(OH)₂D, WT+eldecalcitol, WT+amphotericin B, and WT+a composition of the above vitamin D or analogues thereof and amphotericin B, respectively. The fungal solution was injected into different mice via the tail vein, with 100 μl of the fungal solution injected into each mouse (the amount of fungi used for modeling was 2×10⁵ cells/mouse). The doses of vitamin D₂, vitamin D₃, 25(OH)D, 1,25(OH)₂D, and eldecalcitol were all 0.5 μg/kg; and the experiment started with oral gavage 3 days before infection and ended 30 days after infection. The dose of amphotericin B was 1 mg/kg; and an intraperitoneal injection was administrated 12 h after infection and continued for 7 days.

Survival rate: The survival status of mice in each group was observed and recorded every day. After 30 days of observation, the survival rates were calculated and the surviving mice were killed. Survival curves were plotted and statistically tested using the Log-rank test.

Fungal load in kidneys: On the 3rd and 7th day after model establishment, the left kidneys of each group of mice (3 mice/group) were taken, weighed, and ground. The tissue grinding solutions were diluted and spreaded on YPD plates. The fungal colonies were counted after incubation at 30° C. for 48 h. The results were statistically analyzed using Student t Test.

Kidney function: After the treatment, blood was collected from the mice and levels of creatinine and urea nitrogen were measured using the HITACHI 3100 automatic analyzer.

### (2) Experimental Results

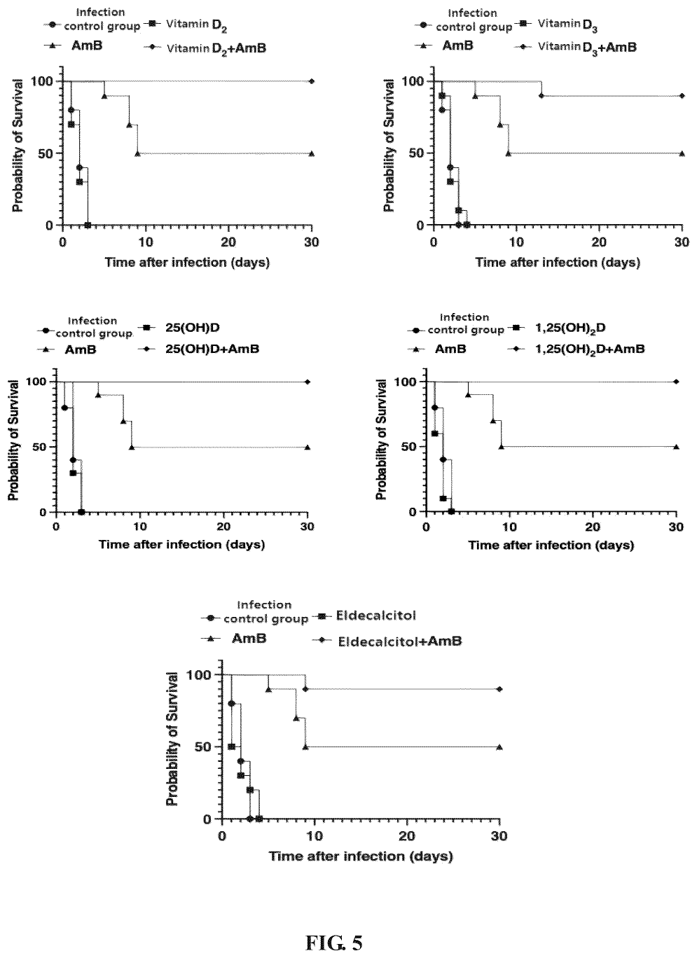

The survival rates are shown in FIG. 5. All mice died within 5 days of systemic Candida albicans infection. Oral administration of vitamin D₂, vitamin D₃, 25(OH)D, 1,25(OH)₂D, and eldecalcitol alone does not significantly improve the survival rates of mice. Compared to the use of amphotericin B alone, the combination of the above vitamin D or analogues thereof with amphotericin B can increase the survival rates of mice from 50%-60% to 90%-100% (P<0.05).

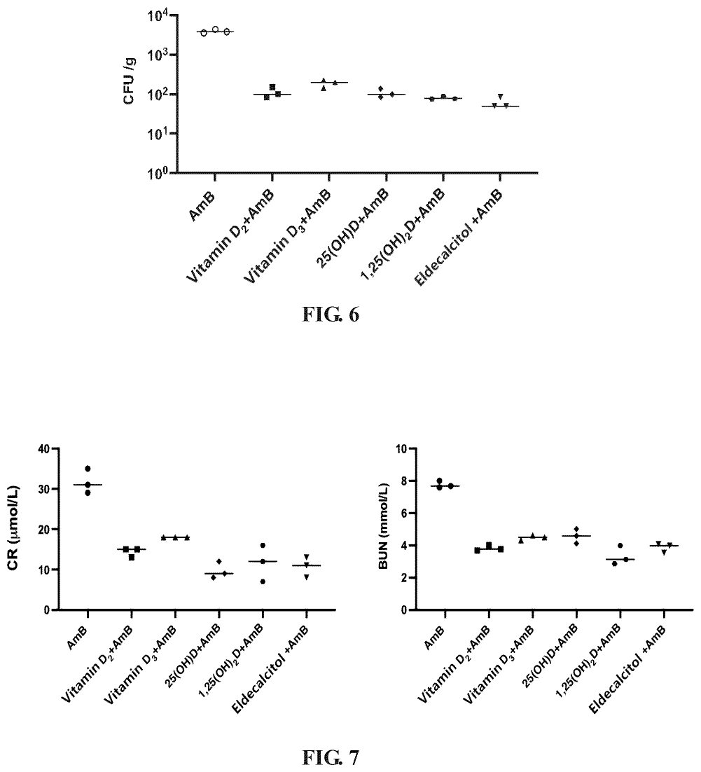

The fungal load in the kidneys is shown in FIG. 6. Since mice in the untreated group and those administrated vitamin D or analogues thereof alone died within 7 days, fungal load detection could not be performed. On the fifth day of treatment, the combination of vitamin D or analogues thereof with amphotericin B reduced the fungal load in the kidneys of mice to about 10² CFU/g when compared with the use of amphotericin B alone (P<0.05).

The results of renal function are shown in FIG. 7. On the fifth day of treatment, the combination of vitamin D or analogues thereof with amphotericin B reduced the plasma creatinine and urea nitrogen levels of mice by approximately 60% when compared with the use of amphotericin B alone.

### Example 7 Efficacy of a Pharmaceutical Composition of Vitamin D or Analogues Thereof and Polyene Drugs in Treating Systemic Fungal Infections in Mice

### (1) Experimental Method

Candida albicans SC5314, Aspergillus fumigatus AF17, and Cryptococcus neoformans ATCC 32609 were activated in YPD liquid medium until the end of logarithmic growth, respectively. The fungal cells were collected and then washed twice with PBS. The fungal solution concentration was adjusted to 2×10⁶ CFU/ml with normal saline. Female BALB/c mice weighing 18-22 g were randomly divided into groups with 10 mice in each group. The mice were grouped into NS (injected with normal saline), WT (injected with SC5314, AF17, or ATCC 32609), WT+vitamin D₂, WT+vitamin D₃, WT+25(OH)D, WT+1,25(OH)₂D, WT+eldecalcitol, WT+amphotericin B, and WT+a composition of the above vitamin D or analogues thereof respectively and amphotericin B, respectively. The drugs were prepared according to the composition preparation method 2 described in Example 1. The fungal solution was injected into different mice via the tail vein, with 100 μl of the fungal solution injected into each mouse (the amount of fungi used for modeling was 2×10⁵ cells/mouse). The doses of vitamin D₂, vitamin D₃, 25(OH)D, 1,25(OH)₂D, and eldecalcitol were all 0.5 μg/kg. The dose of amphotericin B was 1 mg/kg. All drugs were injected intraperitoneally 12 h after infection for 7 consecutive days. The survival status of mice in each group was observed and recorded every day. After 30 days of observation, the survival rate was calculated, the survival curve was drawn, and statistical analysis was performed using the Log-rank test.

### (2) Experimental Results

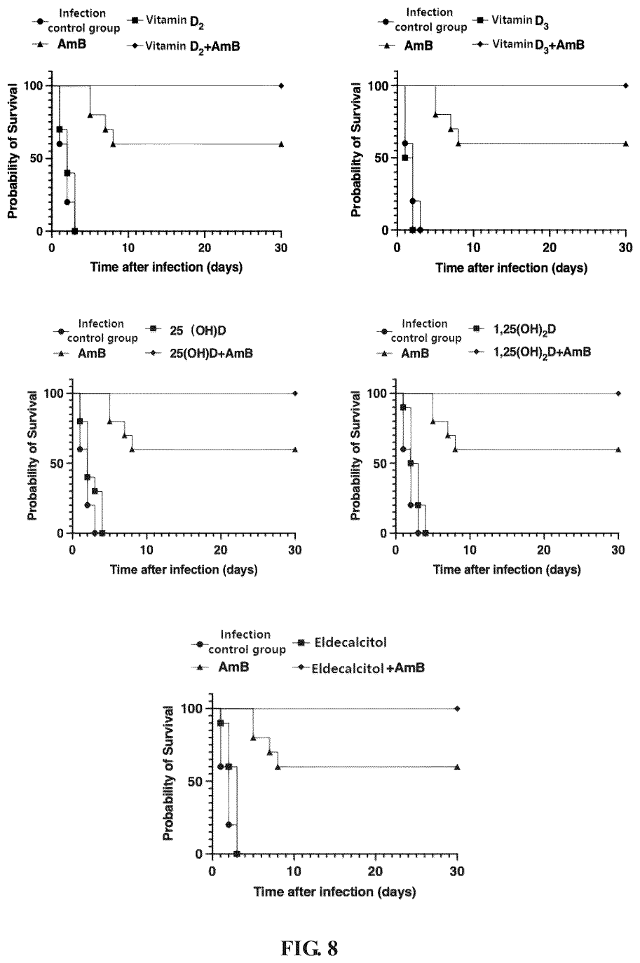

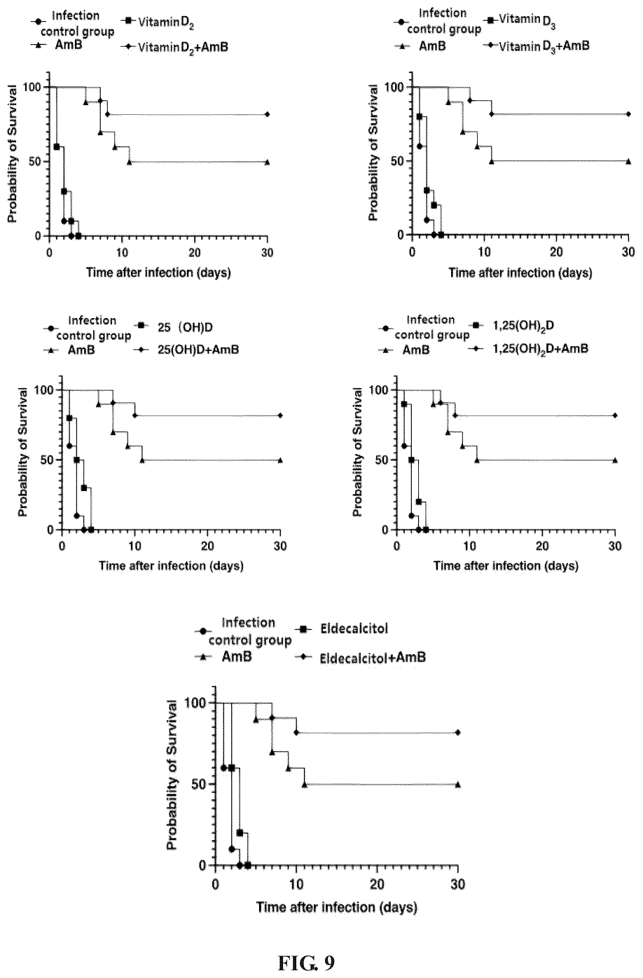

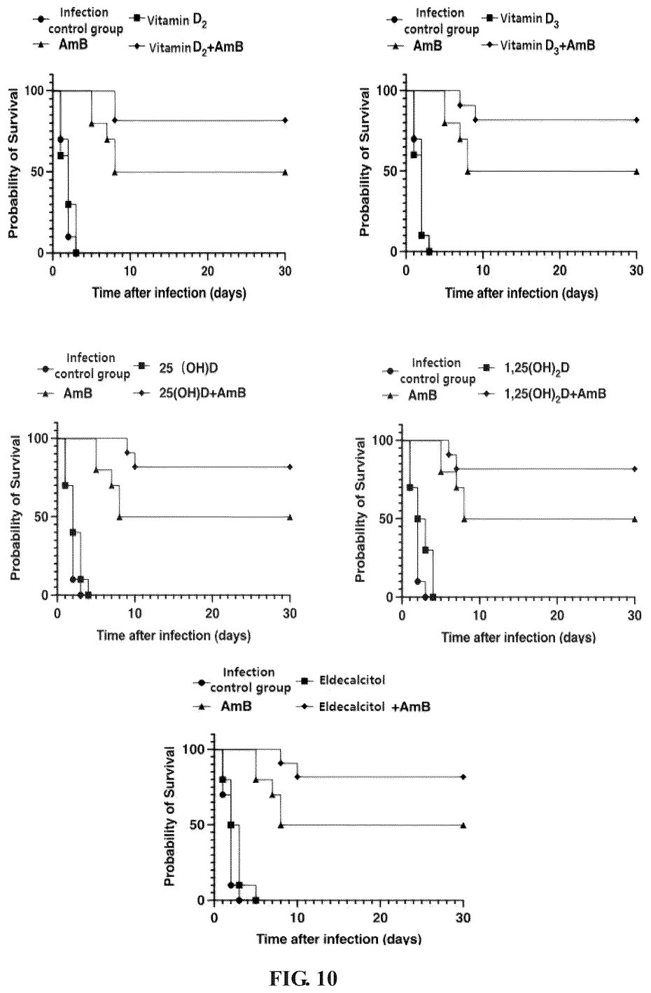

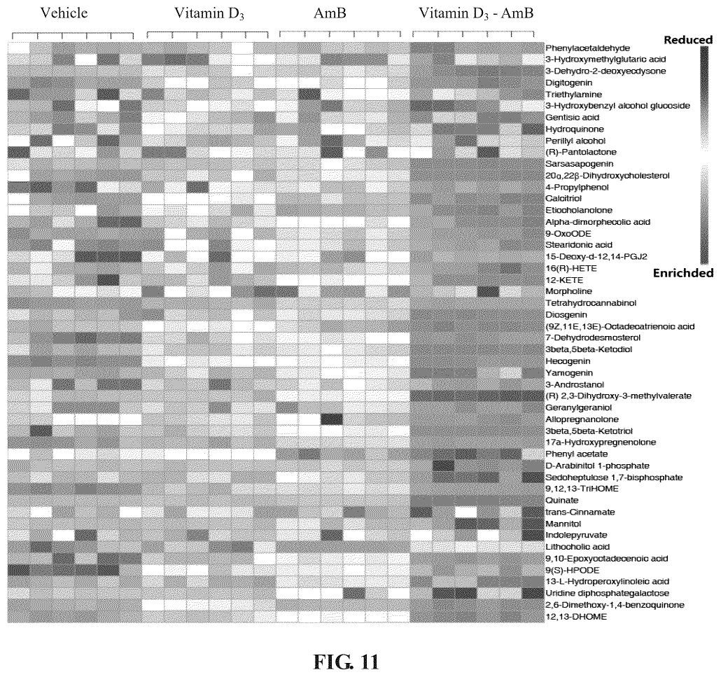

The survival rates of mice infected with Candida albicans, Aspergillus fumigatus, and Cryptococcus neoformans are shown in FIGS. 8, 9, and 10, respectively. Intraperitoneal injection of vitamin D₂, vitamin D₃, 25(OH)D, 1,25(OH)₂D, and eldecalcitol alone does not significantly improve the survival rates of mice. Compared with amphotericin B alone, the pharmaceutical combination can increase the survival rates of mice by 30%-40% (P<0.05).

### Example 8 Mechanisms by which Vitamin D or Analogues Thereof Enhance the Antifungal Efficacy of Polyene Drugs

Taking the combination of vitamin D₃ and amphotericin B as an example, the mechanism by which vitamin D or analogues thereof increase the efficacy of polyene antifungal drugs was clarified through non-targeted metabolomics, reverse transcription-quantitative polymerase chain reaction (RT-qPCR), high performance liquid chromatography (HPLC), isothermal titration calorimetry (ITC) and other methods.

### (1) Experimental Method

a. Using non-targeted metabolomics to detect changes in metabolites within fungal cells after drug action: 70 mg of fungal cells treated with vitamin D₃ (50 μg/ml) and amphotericin B (5 mg/ml) were collected and then mixed with 500 μl of pre-cooled methanol and 500 μl of pre-cooled ultrapure water. The mixture was vortexed for 30 s. 100 mg of glass beads were added to the mixture, which was then frozen in liquid nitrogen for 5 min. The mixture was thawed at room temperature and then oscillated in a grinder at 55 Hz for 2 min. The mixture was centrifuged at 12,000 rpm and 4° C. for 10 min, and the resulting supernatant was dried under vacuum. The dried product was re-dissolved in 300 μl of 2-chlorophenylalanine (4 ppm) methanol-water solution (1:1, 4° C.), the resulting solution was filtered through a 0.22 μm membrane, and the filtrate was analyzed by liquid chromatography-mass spectrometry (LC-MS).
b. Detection of gene transcription levels in the ergosterol biosynthesis pathway using RT-qPCR: RNA of Candida albicans was extracted using the phenol-chloroform method, respectively. Candida albicans was collected after drug (i.e., vitamin D₃ (25 μg/ml) and amphotericin B (2.5 mg/ml)) treatment for 6 h and washed twice with diethylpyrocarbonate (DEPC)-treated water. The obtained Candida albicans was resuspended in 300 μl of pre-cooled acetate-EDTA (AE) buffer, and the resulting solution was mixed with 25 μl of 20 wt % SDS and 300 μl of phenol:chloroform (1:1, v/v). The resulting mixture was mixed at a high speed for 20 seconds and then placed in a 65° C. water bath for 5 minutes. The heated mixture was placed on ice for 10 min and then centrifuged to remove the supernatant. The above steps (from washing with DEPC-treated water to removing the supernatant) were repeated for the obtained precipitate. Subsequently, ETOH+NaAc were added to a microcentrifuge tube containing the precipitate, and then the microcentrifuge tube was stored at −20° C. for 1 h. The mixture in the microcentrifuge tube was washed with 75% ethanol, dried, and then dissolved in ddH₂O and the RNA concentration was measured. After reverse transcription, using cDNA as a template, RT-qPCR was performed with the SYBR primer Premix Ex Taq system and primers for ergosterol synthesis-related genes. The expression of an internal reference gene was used for normalization, and 2^{-ΔΔCt} was calculated.
c. Detection of ergosterol content in whole cells by HPLC: Fungal cells were collected after incubation with drugs (i.e., vitamin D₃ (50 μg/ml) and amphotericin B (2.5 mg/ml)) for 6 h and washed twice with PBS. 3 ml of methanol was added to the microcentrifuge tube containing the fungal cells, and 1 mg of cholesterol as an internal standard was added to the microcentrifuge tube. Glass beads were added to the microcentrifuge tube, and homogenization was performed for 30 seconds. The homogenization was repeated 8-10 times, and the fungal cells in the microcentrifuge tube were incubated at 320 rpm for 1 h. The microcentrifuge tube was centrifuged and the supernatant in the microcentrifuge tube was collected. The collected supernatant was dried under vacuum and resuspended in 500 μl of methanol. Ergosterol and cholesterol standards were prepared and separated on a reverse phase C₁₈ column (250 mm×4 mm) at 30° C., and then eluted in 100% methanol at a flow rate of 1 ml/min. The signals at 282 nm and 210 nm were detected, and the peak areas at 12.5 min and 15 min were recorded to draw a standard curve. Under the same detection conditions, the corresponding peak areas of ergosterol extracts from whole cells and cell membranes were detected and quantified according to the internal standard and standard curve.
d. Detection of the binding affinity of amphotericin B to membrane-embedded ergosterol by isothermal titration calorimetry: A storage solution of palmitoyl-oleoyl-phosphatidylcholine (POPC) (20 mg/ml) and a storage solution of ergosterol (4 mg/ml) were prepared and stored in dry argon at −20° C. After heating the above two solutions to 25° C., they were added to 13×100 mm test tubes, one of which contained 1.2 ml of POPC and 350 μl of ergosterol solution, while the other test tube contained only 1.2 ml of POPC. The solvent in two resulting test tubes was removed in nitrogen flow, and a dried lipid membrane needed to be stored in a high vacuum environment for at least 8 hours before use. Then the obtained lipid membrane was hydrated with 1 ml of K buffer (5 mM HEPES, pH 7.4). The hydrated lipid membrane was subjected to intense vortex for about 3 minutes to form multilamellar vesicles (MLVs). The MLVs were filtered 21 times through a 0.20 μm Millipore (Billerica, MA) polycarbonate filter at Avanti Polar Lipids, forming large unilamellar vesicles (LUVs). The LUV suspension was diluted 10 times with K buffer and added at 10 μl per vial to three 7 ml vials, and the solvent in the vials was removed in nitrogen flow. A vial without lipids was set up as a blank control (the fourth vial), and 450 μl of 8.9 M H₂SO₄ was added to three vials containing dried LUVs and the fourth vial (blank control), respectively. Four samples (vials) were placed in an aluminum block at 225° C. for 25 min and then moved to 23° C. for cooling for 5 min. 150 μl of 30% w/v H₂O₂ aqueous solution was added to the cooled vials, which were then placed in an aluminum block at 225° C. for 30 min and then moved to 23° C. for cooling for 5 min. Then 3.9 ml of water was added to each sample (each vial that had undergone a second cooling step). 500 μl of 2.5% w/v ammonium molybdate was added to each of the resulting vials, and the vials were then subjected to intense vortex five times. After vortexing, 500 μl of 10% w/v ascorbic acid was added to each vial, and then each vial was rotated 5 times. The obtained vials were sealed with polytetrafluoroethylene (PTFE)-lined caps, placed in an aluminum block at 100° C. for 7 min, cooled at 23° C. for 15 min, and then analyzed by ultraviolet/visible (UV/Vis) spectroscopy. The method for determining total phosphorus was to measure the absorbance of a sample at 820 nm and compare this value with the standard curve obtained by this method and the standard phosphorus solution of known concentration to determine the phosphorus concentration. The ergosterol content was determined by spectrophotometry. Three identical independent samples were prepared, including the following steps: adding 50 μl of LUV suspension to 450 μl of a mixture comprising hexane and isopropanol and water with a volume ratio of 2:18:9, and then subjecting the resulting mixture to intense vortex for about 1 minute. The concentration of ergosterol in a solution was detected using UV/Vis spectroscopy (NanoDrop 2000) with an extinction coefficient of 10400 l/mol/cm and a UVmax of 282 nm. Compared to the concentration of phosphorus, the percentage of ergosterol in the solution was determined. An amphotericin B solution (100 μM) or a mixed solution of amphotericin B (50 μM) and vitamin D₃ (50 μM) was added to a sample cell, and LUVs were added to a syringe. The injection method was continuous injections of LUVs, with the first injection of 0.4 μl, followed by 19 subsequent injections. The volume of each subsequent injection was increased by 2 μl from the previous one (i.e., 0.4 μl, 2.4 μl, 4.4 μl . . . ), with a 150-second interval between injections. The experiments were performed at 25° C., and data analysis and titration curve fitting were performed using MicroCal Origin software, assuming a single binding site model, to calculate Kd values. To correct for heats of dilution and mixing, the heat of the last injection of each run was subtracted from all the heats of injections for that particular experiment. Using this method to calculate the total heat change during the experiments, the formula was as follows:

$$Δ {H}_{total} = {\sum }_{i = 1}^{n} ( Δ {h}_{injection}^{i} - Δ {h}_{i n jection}^{n} )$$

where i=number of injections, n=total number of injections, Δh i=heat of the i-th injection, and Δh n=heat of the last injection.

### (2) Experimental Results

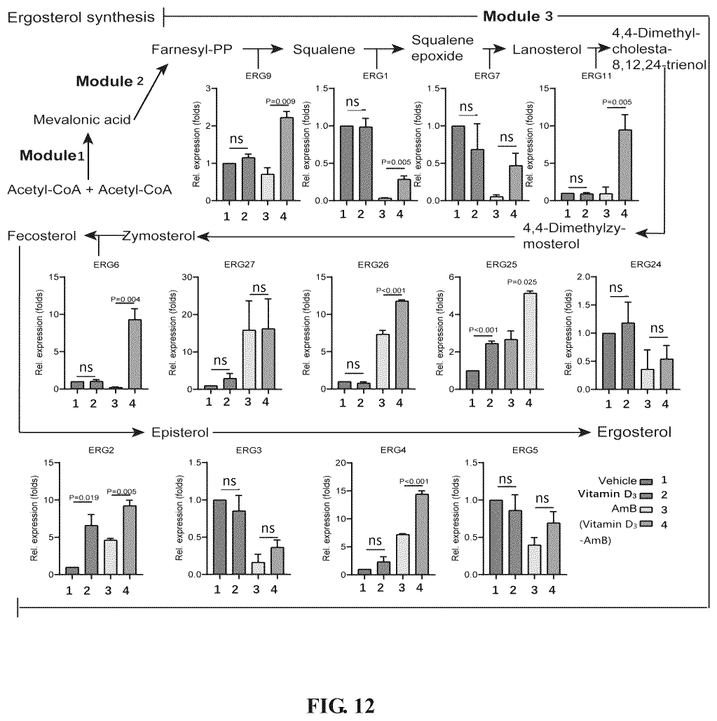

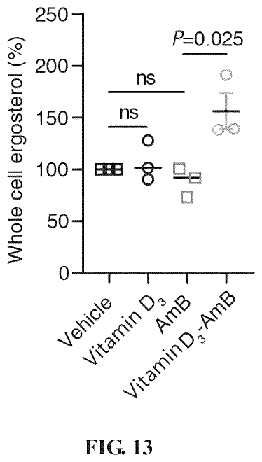

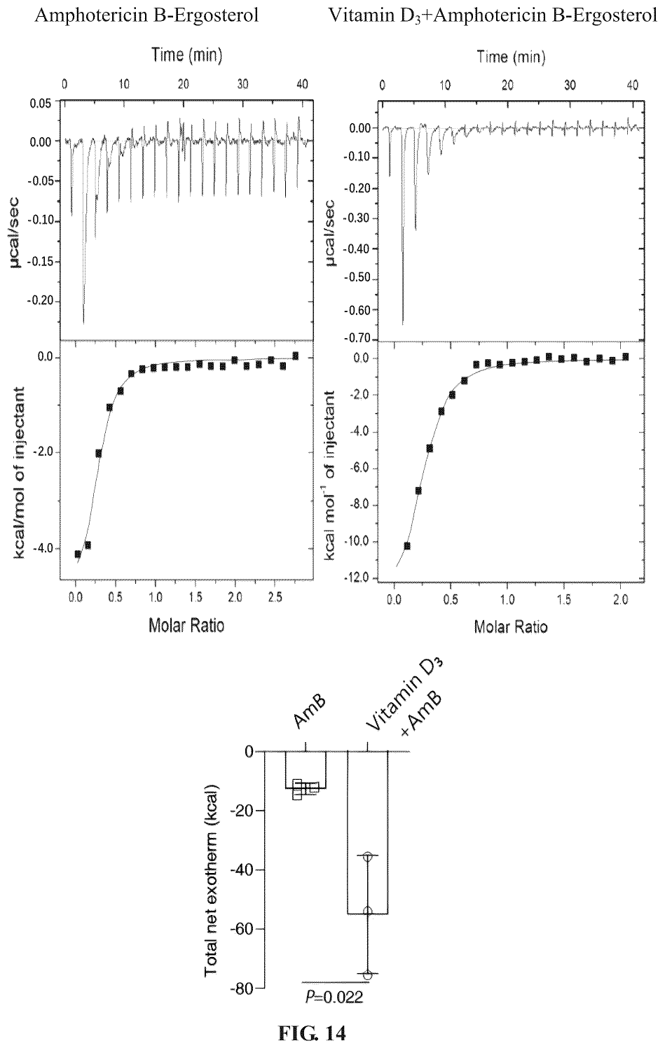

As shown in FIG. 11, compared with the use of amphotericin B alone, the pharmaceutical composition of vitamin D and amphotericin B significantly affects the level of sterol metabolites (e.g., digitogenin, sarsasapogenin, 20α,22β-dihydroxycholesterol, calcitriol, yamogenin, 3-androstanol, and allopregnanolone) in fungal cells. Further, we detected the gene transcription levels in the final stage of the synthesis of ergosterol, the known target of amphotericin B, as well as the content of the final product ergosterol. It is found that compared to the use of amphotericin B alone, this pharmaceutical composition significantly (P<0.05) increases the transcription levels of 8 out of 13 genes (FIG. 12) and the content of the final product ergosterol (FIG. 13). The binding ability of amphotericin B to the target ergosterol was further detected by titration exothermicity. The results show that when vitamin D is combined with amphotericin B, the heat release of the composition binding with ergosterol is more than three times higher than that of amphotericin B alone (FIG. 14). This indicates that the aforementioned pharmaceutical composition enhances the antifungal effect of polyene drugs by increasing the intracellular ergosterol content in fungal cells and enhancing the binding of polyene drugs to ergosterol.

Finally, it is necessary to point out that the above detailed description of the specific embodiments of the present application is only for example. The present application is not limited to the specific embodiments described above. For those skilled in the art, any equivalent modifications and substitutions made to the present application are also within the scope of the present application. Therefore, all equivalent changes and modifications made without departing from the spirit and scope of this application should be included in the scope of this application.

1. An anti-fungal method, comprising: administering an effective amount of component A and an effective amount of component B to a subject in need, the component A is selected from a polyene antifungal drug or a pharmaceutically acceptable salt thereof, and the component B is selected from vitamin D or analogues thereof,
wherein the polyene antifungal drug is selected from amphotericin B or nystatin, and
wherein the vitamin D or analogues thereof are selected from vitamin D₂, vitamin D₃, 25(OH)D, 1,25(OH)₂D, or eldecalcitol.

2. The anti-fungal method of claim 1, wherein the polyene antifungal drug is selected from amphotericin B.

3. The anti-fungal method of claim 1, wherein the component B is selected from vitamin D₃.

4. The anti-fungal method of claim 1, wherein the component B is selected from 25(OH)D.

5. The anti-fungal method of claim 1, wherein the component B is selected from 1,25(OH)₂D.

6. The anti-fungal method of claim 1, wherein the component B is selected from eldecalcitol.

7. The anti-fungal method of claim 2, wherein the component B is selected from vitamin D₃.

8. The anti-fungal method of claim 2, wherein the component B is selected from 25(OH)D.

9. The anti-fungal method of claim 2, wherein the component B is selected from 1,25(OH)₂D.

10. The anti-fungal method of claim 2, wherein the component B is selected from eldecalcitol.

11. The anti-fungal method of claim 1, wherein the fungus is selected from Candida, Aspergillus, Histoplasma, Pneumocystis, Mucor, Cryptococcus, Cladophialophora, Trichophyton, or Microsporum.

12. The anti-fungal method of claim 1, wherein the fungus is selected from Candida albicans, Candida auris, Candida krusei, Aspergillus fumigatus, Histoplasma capsulatum, Pneumocystis carinii, Mucor, Cryptococcus neoformans, Cladophialophora carrionii, Trichophyton rubrum, or Microsporum canis.

13. The anti-fungal method of claim 1, wherein a mass ratio of the component A to component B is 600:1 to 5:1.

14. The anti-fungal method of claim 13, wherein a mass ratio of the component A to component B is 250:1 to 25:1.

15. The anti-fungal method of claim 1, wherein the component A and/or component B further comprises one or more pharmaceutically acceptable carriers.

16. The anti-fungal method of claim 15, wherein the one or more pharmaceutically acceptable carriers are/is selected from liposomes, microcapsules, microspheres, micelles, nanoparticles, lipid complexes, colloidal dispersions, diluents, disintegrants, precipitation inhibitors, surfactants, flow aids, adhesives, lubricants, dispersants, suspending aids, isotonic agents, thickeners, emulsifiers, preservatives, stabilizers, hydrate agents, emulsification enhancers, buffering agents, absorbents, colorants, flavoring agents, sweeteners, corrigents, or antioxidants in the pharmaceutical field.

17. The anti-fungal method of claim 1, the antifungal method being a method for preventing and/or treating a disease caused by fungi.

18. The anti-fungal method of claim 1, wherein an administration method is selected from injection, oral administration, or topical administration.

19. The anti-fungal method of claim 1, wherein the component A and component B are formulated into a pharmaceutical composition comprising the component A and component B.

20. The anti-fungal method of claim 1, wherein the component A and component B are formulated as separate formulations.
